SEQUENCE LISTING (21 sequences)

SEQ ID NO: 1 - DNA, 23 bp, Artificial Sequence
  HuVH1For Forward Primer for variable segment of human antibody heavy chain
caggtgcagctggtgcagtctgg

SEQ ID NO: 2 - DNA, 23 bp, Artificial Sequence
  HuVH2For  Forward Primer for variable segment of human antibody heavy chain
caggtcaacttaagggagtctgg

SEQ ID NO: 3 - DNA, 23 bp, Artificial Sequence
  HuVH3For  Forward Primer for variable segment of human antibody heavy chain
gaggtgcagctggtggagtctgg

SEQ ID NO: 4 - DNA, 23 bp, Artificial Sequence
  HuVH4For  Forward Primer for variable segment of human antibody heavy chain
caggtgcagctgcaggagtcggg

SEQ ID NO: 5 - DNA, 23 bp, Artificial Sequence
  HuVH5For  Forward Primer for variable segment of human antibody heavy chain
gaggtgcagctgttgcagtctgc

SEQ ID NO: 6 - DNA, 23 bp, Artificial Sequence
  HuVH6For  Forward Primer for variable segment of human antibody heavy chain
caggtacagctgcagcagtcagg

SEQ ID NO: 7 - DNA, 23 bp, Artificial Sequence
  HuVH2*For  Forward Primer for variable segment of human antibody heavy chain
cagatcaccttgaaggagtctgg

SEQ ID NO: 8 - DNA, 23 bp, Artificial Sequence
  HuVH4*For  Forward Primer for variable segment of human antibody heavy chain
caggtgcagctacagcagtgggg

SEQ ID NO: 9 - DNA, 24 bp, Artificial Sequence
  HuJH1-2Rev  Reverse Primer for joining segment of human antibody heavy chain
tgaggagacggtgaccagggtgcc

SEQ ID NO: 10 - DNA, 24 bp, Artificial Sequence
  HuJH3Rev  Reverse Primer for joining segment of human antibody heavy chain
tgaagagacggtgaccattgtccc

SEQ ID NO: 11 - DNA, 24 bp, Artificial Sequence
  HuJH4-5Rev  Reverse Primer for joining segment of human antibody heavy chain
tgaggagacggtgaccagggttcc

SEQ ID NO: 12 - DNA, 24 bp, Artificial Sequence
  HuJH6Rev  Reverse Primer for joining segment of human antibody heavy chain
tgaggagacggtgaccgtggtccc

SEQ ID NO: 13 - DNA, 24 bp, Artificial Sequence
  HuJH7Rev  Reverse Primer for joining segment of human antibody heavy chain
tgaccgtggtcccttggccccaga

SEQ ID NO: 14 - DNA, 17 bp, Artificial Sequence
  LMB3  Primer for sequencing
caggaaacagctatgac

SEQ ID NO: 15 - DNA, 18 bp, Artificial Sequence
  HuVHL1For Forward Primer for leader sequence of human antibody heavy chain
crctcctgctgctgacca

SEQ ID NO: 16 - DNA, 19 bp, Artificial Sequence
  HuVHL2For Forward Primer for leader sequence of human antibody heavy chain
grctgagctggrttttcct

SEQ ID NO: 17 - DNA, 19 bp, Artificial Sequence
  HuVHL3For Forward Primer for leader sequence of human antibody heavy chain
krctyygctggsttttyct

SEQ ID NO: 18 - DNA, 19 bp, Artificial Sequence
  HuCDC42bkFor Forward Primer for CDC42 binding kinase
gagcactggccaagcacta

SEQ ID NO: 19 - DNA, 19 bp, Artificial Sequence
  HuCDC42bkRev Reverse Primer for CDC42 binding kinase
tgctctgtgcgagtgtcaa

SEQ ID NO: 20 - DNA, 18 bp, Artificial Sequence
  HuCepsilonRev Reverse Primer for constant region of human antibody epsilon heavy chain
cggatgggctctgtgtgg

SEQ ID NO: 21 - DNA, 243 bp, Homo sapiens
tcagaagcagcggtgagatcctggctgttcctgaaagtgagacgagcggatttcctgctggatggggcgtgcaggttgacactcgcacagagcactggccaagcactaggacgctggagttgcctgcatgggaaggctgacagaggctggtgggctgagtggcagggaagcagctggacaggtgatgcagttccagagagaagcccaaggtgcccagtgtacagctggtatcggatgagatca

(19) United States
(12) Patent Application Publication
(10) Pub. No.: US 2014/0099641 A1
(43) Pub. Date: Apr. 10, 2014

(54) PRIMERS FOR DIAGNOSING ANKYLOSING SPONDYLITIS, AND METHOD FOR DIAGNOSING ANKYLOSING SPONDYLITIS USING THE SAME

(71) Applicants: Chang Hoon Nam, St.Ingbert (DE); Yeon Joo Kim, Saarbruecken (DE); Han-Joo Back, Inchon (KR)

(72) Inventors: Chang Hoon Nam, St.Ingbert (DE); Yeon Joo Kim, Saarbruecken (DE); Han-Joo Back, Inchon (KR)

(73) Assignee: KIST-EUROPE FGMBH, Saarbrucken (DE)

(21) Appl. No.: 13/520,512

(22) PCT Filed: Jan. 6, 2011
(86) PCT No.: PCT/KR11/00095

§ 371 Date: Jul. 3, 2012

(30) Foreign Application Priority Data
Jan. 8, 2010 (KR) 10-2010-0001904
Dec. 17, 2010 (KR) 10-2010-0129848

(51) Int. Cl. C12Q 1/68

(52) U.S. Cl. CPC C12Q 1/6883
USPC 435/6.12; 536/24.33; 536/23.5

(57) ABSTRACT

The present invention relates to primer sets for diagnosing ankylosing spondylitis and the method for diagnosing ankylosing spondylitis using the same. Particularly, the present invention provides primer sets for diagnosing ankylosing spondylitis as follows: (a) a primer set comprising a primer having at least 95% sequence homology with SEQ ID NO: 7, and at least one primer selected from the group consisting of primers of SEQ ID NOs: 9 to 13; (b) a primer set comprising at least one primer selected from the group consisting of primers of SEQ ID NOs: 15 to 17, and a primer having at least 95% sequence homology with SEQ ID NO: 19 and (c) a primer set comprising a primer having at least 95% sequence homology with SEQ ID NO: 18, and a primer having at least 95% sequence homology with SEQ ID NO: 20. The primer sets and the kit for diagnosing ankylosing spondylitis of the present invention can be effectively used for early diagnosis. tracking progress and prognosis of ankylosing spondylitis.

### TECHNICAL FIELD

The present invention relates to primers for diagnosing ankylosing spondylitis and the method for diagnosing ankylosing spondylitis using the same.

### BACKGROUND ART

Ankylosing spondylitis (AS), a type of severely progressed Spondyloarthropathy (SpA), is a chronic progressive systemic disease characterized in that it starts in late teenagers to early twenties and shows symptoms such as typical spondylosis, chronic inflammation of the sacroiliac joints and spin, the invasion to peripheral joints, eyes, heart, or intestine in thirties to forties.

The prevalence rate of SpA depends on each country, however, it is usually about 1-2%. SpA restricts the activities of patients and thus it results in the negative effects on society and economics. Recently, according to the accumulation of new knowledge about the progress and treatment of SpA, it has been revealed that early treatment is effective for the treatment and inhibiting the progress of SpA, and thus the early diagnosis has become an important issue.

The cause of SpA has not been clearly revealed, however, it is presumed that genetic abnormalities, infections, and/or immune inflammatory responses may cause the disease like systemic rheumatic diseases such as rheumatoid arthritis or systemic lupus erythematosus. Genetically, it is presumed that ‘HLA-B27’ gene, histocompatibility antigen, is involved in the outbreak of the disease. Additionally, recent researches suggest that ERAP1 (endoplasmic reticulum aminopeptidase 1) and IL23R (interleukin 23 receptor) genes are related to the disease. Waist and sacral pain caused by ankylosing spondylitis start slowly, and arthritis in knees and ankles is accompanied in some cases. It is difficult to distinguish from common backache or non specific arthritis pain because the symptom is vague and deterioration of symptoms and improvement is repeated. According to the research in Europe, it takes about 10 years from onset of SpA symptoms to the diagnosis of AS. Therefore, it is frequently too late to treat the disease when a patient sees a doctor.

The classical diagnosis of AS is conducted on the basis of the patient's symptoms, medical history, and radiological findings of sacroiliac joints and spine. Especially, abnormalities in radiological findings are shown only when the disease is sufficiently progressed and thus early diagnosisis impossible by radiological findings. Recently, MRI scan is used for early diagnosis. However, this test is restrictively used because it is expensive and time consuming. ‘HLA-B27’ gene test in blood is also being used for early diagnosis, but negative results in 15% of patients and false positive results in a considerable number of normal patients are showed. In addition, HLA-B27 does not provide any information about prognosis and tracking progress of the disease.

Furthermore, more inflammatory markers such as ESR (erythrocyte bloodsedimentationrate) or CRP (C reactive protein) does not reflect AS activity while these markers are generally used as disease activity index in many rheumatic diseases. Thus, the response to treatment and progress of AS are still evaluated by patients subjective explanation. Meanwhile, rheumatoid factor or anti-cyclic citrullinated peptide antibody found in rheumatoid arthritis patients is used as a prognosis index. In other words, the positive index indicates the outbreak and fast progression of arthritis, thus, early and aggressive treatment are required for the patient with positive index. However, there is no prognosis index for AS.

In conclusion, there is no biomarker for early diagnosis of AS except for the limited use of HLA-B27. In addition, there is no biomarker to reflect the prognosis of AS or activity index for tracking progress in AS. Therefore, discovering of biomarkers for early diagnosis, prognosis, and/or tracking progress of AS are enormous.

### DISCLOSURE OF INVENTION

### Technical Problem

The object of the present invention is to provide an early diagnosis method of AS. Particularly, the present invention is to provide primer sets, kits, and a biomarker for diagnosing AS.

### Solution to Problem

One aspect of the present invention provides primer sets for diagnosing AS. In one embodiment of the present invention, there is provided a primer set comprising as follows: (a) a forward primer having at least 95% sequence homology with SEQ ID NO: 7 and (b) at least one reverse primer selected from the group consisting of primers of SEQ ID NOs: 9 to 13.

In another embodiment of the present invention, there is provided a primer set comprising as follows: (a) at least one forward primer selected from the group consisting of primers of SEQ ID NOs: 15 to 17 and (b) a reverse primer having at least 95% sequence homology with SEQ ID NO: 19.

In another embodiment of the present invention, there is provided a primer set comprising as follows: (a) a forward primer having at least 95% sequence homology with SEQ ID NO: 18 and (b) a reverse primer having at least 95% sequence homology with SEQ ID NO: 20.

Another aspect of the present invention provides a method for diagnosing ankylosing spondylitis using the above primer sets. In one embodiment of the present invention, the method may comprise the following steps: (a) synthesizing cDNA by using biological samples of patients; and (b) performing PCR (polymerase chain reaction) by using the above synthetic cDNA and a primer set having at least 80% sequence homology with any one of primer sets of the present invention, and (c) determining nucleic acid sequences of the PCR product.

Another aspect of the present invention provides a kit for diagnosing AS comprising a primer set having at least 80% sequence homology with any one of primer sets of the present invention.

Another aspect of the present invention provides a biomarker for diagnosing AS comprising total or a part of sequence of SEQ ID NO: 21.

Another aspect of the present invention provides a method for diagnosing AS using total or a part of nucleic acid sequence of SEQ ID NO: 21.

In one embodiment of the present invention, the method may comprise following steps: (a) synthesizing cDNA by using biological samples of patients; (b) performing PCR (polymerase chain reaction) using the above synthetic cDNA and a primer set having at least 80% sequence homology with any one of primer sets of the present invention; and (c) identifying total or a part of sequence of SEQ ID NO: 21 in the PCR product.

Another aspect of the present invention provides a kit for diagnosing AS comprising total or a part of nucleic acid sequence of SEQ ID NO: 21. In one embodiment of the present invention, the kit may comprise a nucleic acid having at least 80% sequence homology with the nucleic acid sequence of SEQ ID NO: 21

### Advantageous Effects of Invention

The present invention provides primer sets for diagnosing AS and the method for diagnosing AS using the same. The primer sets and the kit for diagnosing ankylosing spondylitis of the present invention can be effectively used for early diagnosis, tracking progress and prognosis of AS.

### BRIEF DESCRIPTION OF DRAWINGS

FIG. 1 is the result of PCR showing the difference in expression patterns between normal control groups (NO) and AS patients groups (AS) when HuVH2*For was used as a forward primer.

FIG. 2 shows the result of colony polymerase chain reaction for screening strains transformed with recombinant DNA (N: colony number, A: selected colonies, B: excluded colonies showing weak bands).

FIG. 3 shows the sequence of VH2* fragment normally inserted into E. coli.

FIG. 4 is a diagram showing the location of CDC42 BPB (binding protein kinase beta) gene and immunoglobulin gene on chromosome 14.

FIG. 5 is a diagram showing the genetic map of CDC42 BPB (binding protein kinase beta).

FIG. 6 is a diagram showing a regular pattern of antibody fragment in AS patient group.

FIG. 7 shows antibody fragments copied by primer sets of the present invention.

FIG. 8 shows the result of quantitativegroup 1 primer sets.

FIG. 9 shows the average of each group of quantitativegroup 1 primer sets.

FIG. 10 shows the result of each sample of quantitativegroup 1 primer sets.

FIG. 11 shows the result of quantitativegroup 2 primer sets.

FIG. 12 shows the average of each group of quantitativegroup 2 primer sets.

FIG. 13 shows the result of each sample of quantitativegroup 2 primer sets.

FIG. 14 shows the result of quantitativegroup 3 primer sets.

FIG. 15 shows the average of each group of quantitativegroup 3 primer sets.

FIG. 16 shows the result of each sample of quantitativegroup 3 primer sets.

### BEST MODE FOR CARRYING OUT THE INVENTION

The present inventors have studied a new method for diagnosing AS and have found that primer sets of the present invention produce a specific PCR product in patients with AS.

The present invention provides primer sets for diagnosing AS. Primer sets of the present invention produce a specific PCR product in patients with AS, which is not produced in non-AS patients.

In the present invention, “primer” is a single stranded oligonucleotide sequence complementary to the nucleic acid which is copied, and it serves as a starting point for primer elongation product. There is no specific limitation in length and sequence of the primer if the primer enables the start of synthesis. Preferably, the length of primer is about 5-50 nucleotides. Specific length and sequence of the primer depend on complexity of DNA or RNA targets, and conditions such as temperature and ionic strength.

Primer sets of the present invention may amplify specifically expressed genes in patients with AS. The primer sets of the present invention may comprise as follows:

(1) a forward primer having at least 95% sequence homology with SEQ ID NO: 9, and at least one reverse primer selected from the group consisting of primers of SEQ ID NOs: 9 to 13

(2) at least one forward primer selected from the group consisting of primers of SEQ ID NOs: 15 to 17, and a reverse primer having at least 95% sequence homology with SEQ ID NO: 19 and

(3) a forward primer having at least 95% sequence homology with SEQ ID NO: 18, and a reverse primer having at least 95% sequence homology with SEQ ID NO: 20.

The primers according to the present invention comprise the functional equivalents having at least 80%, preferably 90%, sequence homology with each primer of the present invention. The functional equivalents produce substantial equivalents with PCR products which are specifically produced in AS patients when primers of the present invention are used The functional equivalents can be generated a result of an addition, substitution, or deletion of a part of the present primer sequences. Preferably, the substitution may be a conservative substitution.

Oligonucleotides used as a primer of the present invention may comprise intercalating agents or nucleotide analogues such as phosphorothioate, alkylphosphorothioate, peptide nucleic acid.

In addition, indicators to provide a signal may be attached to the primer of the present invention. The indicator may be substances emitting fluorescence, phosphorescence, or radiation, however, there is no limitation on the indicator. Preferably, the indicator may be Cy-5 or Cy-3.

Primer sets of the present invention may be efficiently used for early diagnosis of AS. Therefore, the present invention provides a method for diagnosing AS using the present primer sets. In one embodiment of the present invention, the method may comprise following steps: (a) synthesizing cDNA by using biological samples of patients; (b) performing PCR (polymerase chain reaction) by using the above synthetic cDNA and a primer set having at least 80% sequence homology with any one of primer sets of the present invention; and (c) determining nucleic acid sequences of the PCR product.

In step (a), the biological samples may comprise saliva, biopsy, blood, skin tissue, liquid cultures, feces, and urine. Preferably, it may be blood. More preferably, it may be peripheral blood. The cDNA synthesis may be performed by commonly used methods in the art or commercially available cDNA production kits.

In step (b), PCR is performed using cDNA from step (a) and primer sets of the present invention. The PCR may be performed using PCR reaction mixture containing multiple components known in the art. The PCR reaction mixture may comprise a proper amount of DNA polymerase, dNTP, PCR buffer solution, and water (dH2O). The PCR buffer solution may comprise Tris-HCl, MgCl₂, and KCl. The concentration of MgCl₂ significantly affects extension specificity and yield. For example, nonspecific PCR products are increased when the concentration of Mg²⁺ is too high while yield of PCR products is decreased when the concentration of Mg²⁺ is too low. Preferably, the concentration of MgCl₂ may be 1.5-2.5 mM. The PCR buffer solution may additionally comprise a proper amount of Triton X-100. In addition, PCR may be performed according to general PCR reaction condition: pre-denaturation of template DNA at 94-95° C. denaturation annealing extension and elongation at 72° C.

The denaturation and extension may be performed at 94° C.-95° C. and 72° C. respectively. And annealing temperature can vary according to the type of primer. Preferably, annealing temperature is 52° C.-65° C., and more preferably the temperature is 60° C. The number of cycle and time of each step may be determined by general conditions in the art. According to the present invention, the preferable PCR reaction condition is as follows: (1) pre-denaturation of template DNA at 95° C. for 3 minutes (2) 30 cycles of following steps: 30 seconds at 94° C., 30 seconds at 60° C., and 1 minute at 72° C. and (3) 5 minutes at 72° C.

Another aspect of the present invention provides a kit for diagnosing AS comprising a primer set of the present invention. The diagnosing methods are the same as those described in the above. The kit may additionally comprise components necessary to perform the electrophoresis to identify extension of PCT products. And the kit may comprise PCR reaction buffer and DNA polymerase to easily perform PCR reactions easily.

The known methods described in the above may be found in the following references (Maniatis et al., Molecular Cloning: A Laboratory Manual, Cold Spring Harbor Laboratory, Cold Spring Harbor, N.Y. (1982); Sambrook et al., Molecular Cloning: A Laboratory Manual, 2d Ed., Cold Spring Harbor Laboratory Press (1989); Deutscher, M., Guide to Protein Purification Methods Enzymology, vol. 182. Academic Press. Inc., San Diego, Calif. (1990)).

In one experiment of the present invention, cDNA was synthesized using blood of AS patients and healthy human beings, and primers producing specific PCR products in AS patients were selected through PCR. As a result, specific PCR products were identified in AS patients when the primer of SEQ ID NO: 7 was used as a forward primer, and a mixture of the primers of SEQ ID NOs: 9 to 13 was used as a reverse primer (see Exp. 1).

In another experiment of the present invention, the PCR product from experiment 1 was amplified through the following steps: inserting the product into PIT2phagemid vector, inserting the vector into E. coli by electrical injection, and incubating E. coli. (see Exp. 2).

In another experiment of the present invention, E. coli strains successfully transfected with the specific PCR product were selected, DNA was isolated, and nucleic acid sequences were obtained (see Exp. 3).

In another experiment of the present invention, the nucleic acid sequence (from Exp. 3) was identified through gene database searches and it was revealed that the sequence corresponds to a part of intron sequences of CDC 42 BPB (binding protein kinase beta) (designated as SEQ ID NO:21). In addition, same PCR products with specific nucleic acid sequence were confirmed in blood samples of 39 AS patients. From these results, it is expected that the sequence of SEQ ID NO: 21 may be a biomarker for diagnosing AS.

Therefore, another aspect of the present invention provides a method for diagnosing of AS using total or a part of sequence of SEQ ID NO: 21.

In one embodiment of the present invention, the method may comprise following steps: (a) synthesizing cDNA by using biological samples of patients; (b) performing PCR (polymerase chain reaction) using the above synthetic cDNA and a primer set having at least 80% sequence homology with any one of primer sets of the present invention; and (c) identifying total or a part of sequence of SEQ ID NO: 21 in the PCR product.

The present inventors additionally established a primer set which produces a specific PCR product in AS patients based on the nucleic acid sequence of specific PCR products form Experiment 4 (see Exp. 5).

In the following, the present invention is described in detail through experiments. The experiments are not intended to limit the technical spirit of the present invention, but are intended to describe the invention.

### Experiment 1

### Extension of VH Genes

After collecting blood from AS patients and normal control groups, PBMC (peripheral blood mononuclear cells) and serum were extracted using a density gradient centrifugation of isolation medium (Histopaque-sigma, UK), and cDNA was synthesized. Then, VH (immunoglobulin heavy chain variable region) genes of each samples were extended from the synthesized cDNA using VH forward primer and JH (immunoglobulin heavy chain joining region) reverse primer. The primers were designed on the basis of typically used primers to extend human VH genes from cDNA (Van et al., (2003) Clin. Exp. Immunol. 131(2):364-376), and three kinds of primer were added through comparison with Immunoglobulin Blast Human VH germline gene sequences. (primers of SEQ ID NO: 6, SEQ ID NO: 7, and SEQ ID NO: 13)

TABLE 1
Primers used in extending human VH from cDNA

| SEQ | | |
|---|---|---|
| ID NO: | name | Sequence (5′->3′) |
| 1 | HuVH1For | CAG GTG CAG CTG GTG CAG TCT GG |
| 2 | HuVH2For | CAG GTC AAC TTA AGG GAG TCT GG |
| 3 | HuVH3For | GAG GTG CAG CTG GTG GAG TCT GG |
| 4 | HuVH4For | CAG GTG CAG CTG CAG GAG TCG GG |
| 5 | HuVH5For | GAG GTG CAG CTG TTG CAG TCT GC |
| 6 | HuVH6For | CAG GTA CAG CTG CAG CAG TCA GG |
| 7 | HuVH2*For | CAG ATC ACC TTG AAG GAG TCT GG |
| 8 | HuVH4*For | CAG GTG CAG CTA CAG CAG TGG GG |
| 9 | HuJH1- | TGA GGA GAC GGT GAC CAG GGT GCC |
| | 2Rev | |
| 10 | HuJH3Rev | TGA AGA GAC GGT GAC CAT TGT CCC |
| 11 | HuJH4- | TGA GGA GAC GGT GAC CAG GGT TCC |
| | 5Rev | |
| 12 | HuJH6Rev | TGA GGA GAC GGT GAC CGT GGT CCC |
| 13 | HuJH7Rev | TGA CCG TGG TCC CTT GGC CCC AGA |

[A is a(Adenine); C is c(Cytosine); G is g(Guanine); T is t(Thymine); U is u(Uracil); Y is c or t(u); R is a or g; M is a or c; K is g or t(u); S is g or c; W is a or t(u); H is a or c or t(u); B is g or t(u) or c; V is g or c or a; D is g or a or t(u); N is g, a, c or t(u).]

Fragments of the VH region were obtained from cDNA samples of AS patients and healthy controls through PCR with the following conditions. For 50 μl of entire reaction mixture, 2.5 μl of each VH forward primer (HuVH1For, HuVH2For, HuVH3For, HuVH4For, HuVH5For, HuVH6For, HuVH2*For, or HuVH4*For) and 2.5 μl of reverse primer mixture (mixture of same concentration of HuJH1-2Rev, HuJH3Rev, HuJH4-5Rev, HuJH6Rev, HuJH7Rev) were added, and then, 1 μl of dNTP, 1 μl of sample DNA, and 1 μl of PCR polymerase were added and mixed. After that, sample DNA was extended through 30 cycles of following steps: 30 seconds at 94° C. 30 seconds at 60° C. and 1 minute at 72° C. After the agarose gel electrophoresis of amplified DNA, the DNA of predicted size was purified by heat extraction techniques.

As a result, there was no difference in the expression pattern between normal control groups and AS patients groups when HuVH1For, HuVH2For, HuVH3For, HuVH4For, HuVH5For, HuVH6For, or HuVH4*For was used as a forward primer. However, it was confirmed that there is difference in expression patterns between normal control groups and AS patients groups when HuVH2*For was used as a forward primer (see FIG. 1). Finally, antibody fragments from normal control groups and AS patients groups were obtained (VH1, VH3, and VH5 fragments from normal control groups; VH1, VH3, VH5, and VH2* fragments from AS patients groups.)

### Experiment 2

### Transformation of Bacteria with VH2*

VH2* fragment obtained in experiment 1 and PIT2 phagemid vector were mixed with 40 μl of restriction enzyme NcoI in 100 μl of total reaction mixture. After incubation at 37° C. for 4 hours and purification, 4 μl of restriction enzyme XhoI was added and incubated at 37° C. for 4 hours, agarose gel electrophoresis was performed, and DNA was isolated by heat extraction techniques.

PIT2 phagemid vector was treated with NcoI and XhoI, incubated at 37° C. for 1 hour after adding 2 μl of phosphatase, and purified. 3 μl of VH2* fragments cutted by restriction enzymes and 4 μl of PIT2 phagemid vector treated with restriction enzymes and phosphatase were added in 20 μl of total reaction mixture, 1 μl of ligase was mixed with them, and the reaction mixture was incubated at 16° C. for 18 hours. The incubated reaction mixture was purified and inserted to E. coli TG1 strain using electroporation.

The reaction condition of electroporation was 12.5 kV/cm, 200Ω, and 25 mF. After electroporation, E. coli was spread on agar plates containing ampicillin, and incubated at 37° C. for 16 hours.

### Experiment 3

### Sequence Analysis of Transformed Strains

Strains transformed with recombinant DNA obtained from experiment 2 were grown in agar plates containing ampicillin and selected. After selecting and incubating the transformed strains in LB medium for 16 hours, recombinant DNA in the transformed strains was purified and sequenced using LMB3 primer (SEQ ID NO: 14; 5′-CAG GAA ACA GCT ATG AC-3′ to identify VH2* fragment in recombinant DNA.

Through DNA sequencing of clones which were selected by colony polymerase chain reaction, it was confirmed that VH2* fragment was inserted into the recombinant DNA. Colony polymerase chain reaction was performed in the same PCR conditions by using a primer set for VH2* extension in above experiment 1, and colonies grown in media containing ampicillin were selected and used as template DNA.

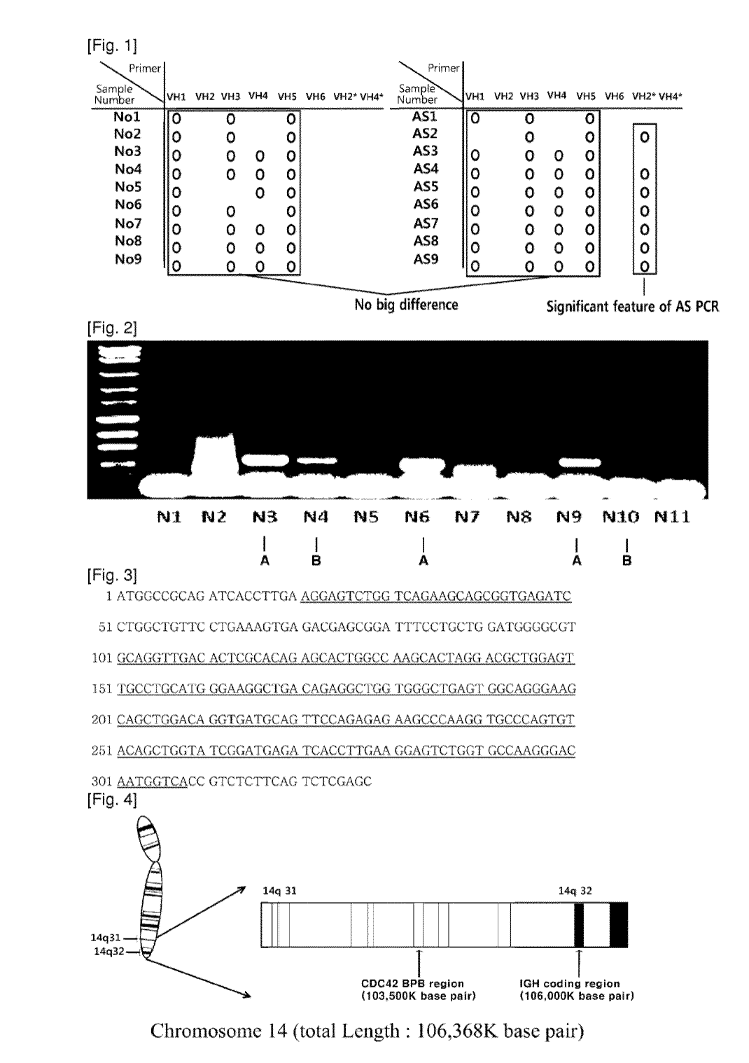

After electrophoresis of PCR products, clones with predicted size of DNA were selected. As shown in FIG. 2, clones with clear DNA band were N (colony number) 3, 6, and 9 (represented by A), and N4 and N 10 with a weak band (represented by B) were excluded. NA sequences of colonies selected by colony polymerase chain reaction were obtained [performed by Eurofins MWG Operon (Germany)], and the obtained DNA sequences were analyzed using Vector NTI Suite 6 program (Invitrogen, USA). As a result, 48% of total selected clones showed specific continued sequence (see FIG. 3.)

### Experiment 4

### Identification of Biomarker for AS

The specific continued sequence obtained from experiment 3 was identified using nucleotide sequence database (GenBank, EMBL and RefSeq).

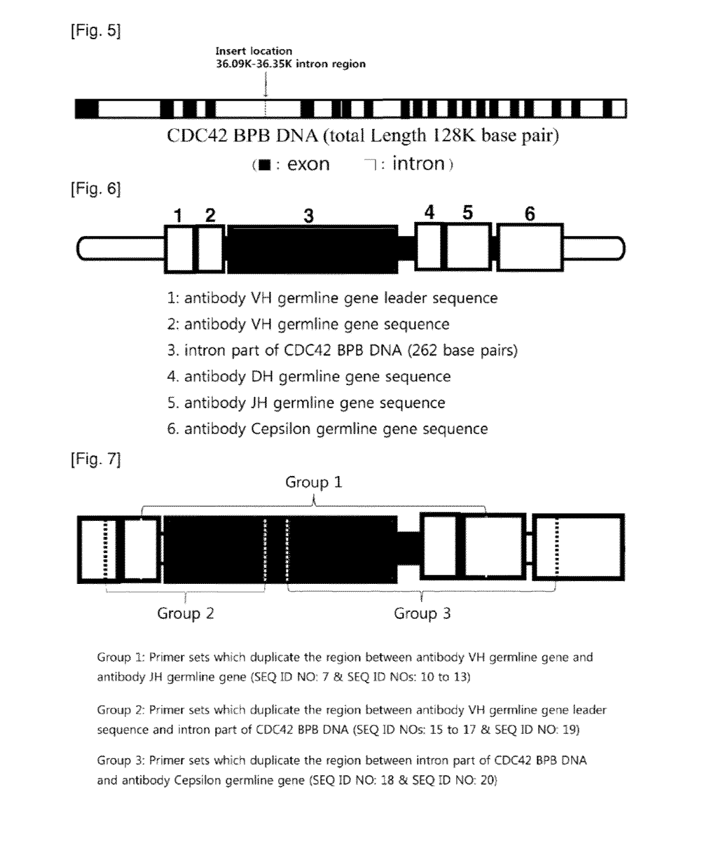

As a result, it was revealed that VH2* fragment of the present invention corresponds to the intron part of CDC42 BPB (binding protein kinase beta). CDC42 BPB is located at chromosome 14 (4q32 83660K) and total length is 1278K bps (see FIG. 4), the specific continued sequence obtained from experiment 3, which corresponds to the intron part of CDC42 BPB and the length is between 36.09K bps and 36.35K bps (see FIG. 5), was designated as SEQ ID NO:21.

In addition, the similarity between nucleotide sequences of VH2* fragments obtained from AS patient group were examined. After synthesizing cDNA from blood samples of 9 AS patients, PCR was performed using SEQ ID NO: 7 as a forward primer, and primer mixtures with same concentration of SEQ ID NO: 9 to 13 as a reverse primer. Nucleotide sequences were obtained according to the same method of experiment 2 and experiment 3, and DNA sequences were analyzed by Vector NTI Suite 6 program (Invitrogen, USA).

As a result, it was confirmed that the sequence of VH2* fragment conservatively exist in AS patient group. In addition, as shown in FIG. 6, it was revealed that VH2* fragment in AS patient group is interlocked with the nucleotide sequence of CDC42 BPB in a regular pattern (see FIG. 6 showing a regular pattern of antibody fragment in AS patient group).

### Experiment 5

### Additional Establishment of Primer Sets for Diagnosing AS

From the structure revealed in Example 4, it was confirmed that a part of intron of CDC42 BPB is inserted into VH2 antibody gene by chromosomal inversion. Based on this result, the present inventor made primers from variable region leader sequence (VH-L) of antibody heavy chain, intron of CDC42 BPB, and the constant region of antibody. Therefore, additional primer sets produce specific PCR products in AS patients were established.

TABLE 2
Additional primer sets for diagnosing AS

| SEQ | | |
|---|---|---|
| ID NO: | Name | Sequence (5′->3′) |
| 15 | HuVHL1For | CR CTC CTG CTG CTG ACC A |
| 16 | HuVHL2For | GR CTG AGC TGG WIT TTC CT |
| 17 | HuVHL3For | KR CTY YGC TGG STT TTY CT |
| 18 | HuCDC42BPBFor | GAG CAC TGG CCA AGC ACT A |
| 19 | HuCDC42BPBRev | TG CTC TGT GCG AGT GTC A A |
| 20 | HuCepsilonRev | CGG ATG GGC TCT GTG TGG |

The additional primer sets are as follows: (1) a primer set comprising at least one primer selected from the group consisting of primers of SEQ ID NOs: 15 to 17, and a primer of SEQ ID NO: 19; and (2) a primer set comprising a primer of SEQ ID NO: 18 and a primer of SEQ ID NO: 20.

The primer sets of the present inventions are shown in Table 3.

TABLE 3
Primer Sets

| | SEQ ID | | |
|---|---|---|---|
| Group | NO: | Name | Sequence (5′->3′) |
| 1 | 7 | HuVH2*For | CAG ATC ACC TTG AAG GAG TCT GG |
| | 9 | HuJH1-2Rev | TGA GGA GAC GGT GAC CAG GGT GCC |
| | 10 | HuJH3Rev | TGA AGA GAC GGT GAC CAT TGT CCC |
| | 11 | HuJH4-5Rev | TGA GGA GAC GGT GAC CAG GGT TCC |
| | 12 | HuJH6Rev | TGA GGA GAC GGT GAC CGT GGT CCC |
| | 13 | HuJH7Rev | TGA CCG TGG TCC CTT GGC CCC AGA |
| 2 | 15 | HuVHL1For | CR CTC CTG CTG CTG ACC A |
| | 16 | HuVHL2For | GR CTG AGC TGG RTT TTC CT |
| | 17 | HuVHL3For | KR CTY YGC TGG STT TTY CT |
| | 19 | HuCDC42BPBRev | TG CTC TGT GCG AGT GTC A A |
| 3 | 18 | HuCDC42BPBFor | GAG CAC TGG CCA AGC ACT A |
| | 20 | HuCepsilonRev | CGG ATG GGC TCT GTG TGG |

The antibody fragments copied by primer sets of the present invention are shown in FIG. 7. As shown in FIG. 7, the region between antibody VH germline gene and antibody JH germline gene is copied by group 1 primer sets, the region between antibody VH germline gene leader sequence and intron part of CDC42 BPB DNA is copied by group 2 primer sets, and the region between intron part of CDC42 BPB DNA and antibody Cepsilon germline gene is copied by group 3 primer sets.

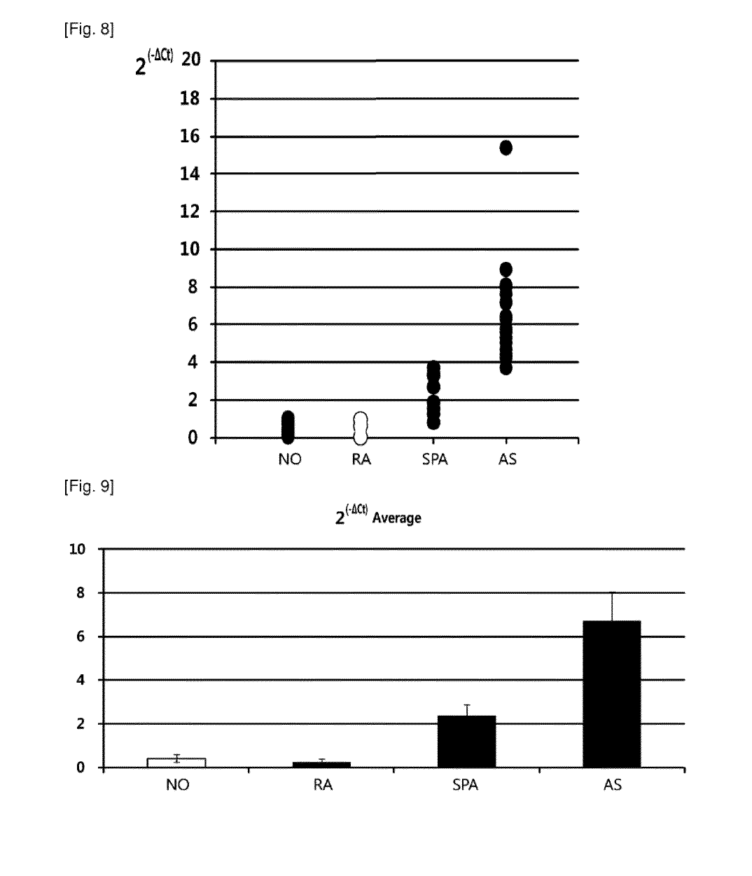

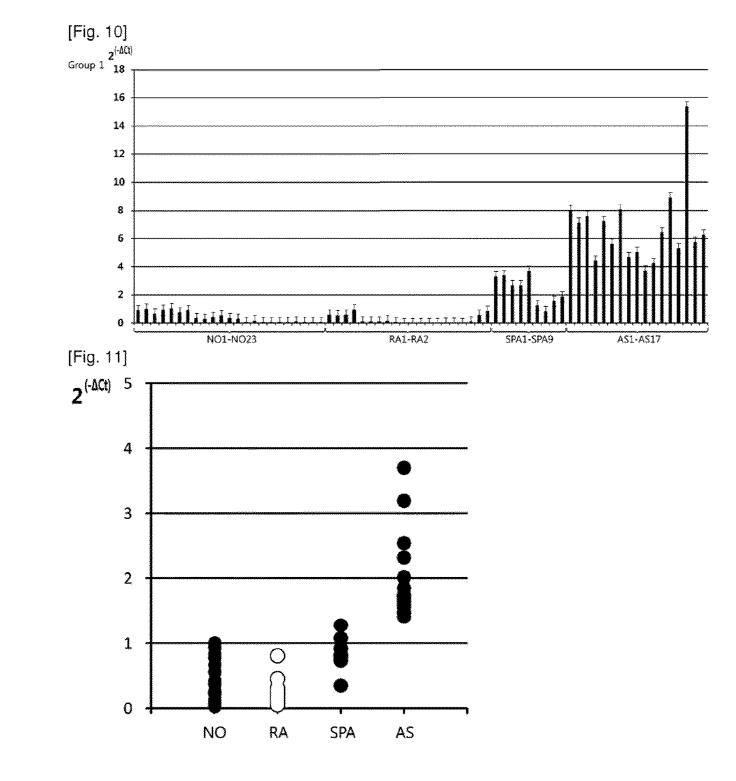

FIG. 8 shows the result of quantitativegroup 1 primer sets, FIG. 9 shows the average of each group of quantitativegroup 1 primer sets, and FIG. 10 shows the result of each sample of quantitative using group 1 primer sets (NO: normal control group, RA: rheumatoid arthritis patient group, SPA: Spondyloarthropathy patient group, AS: Ankylosing spondylitis patient group).

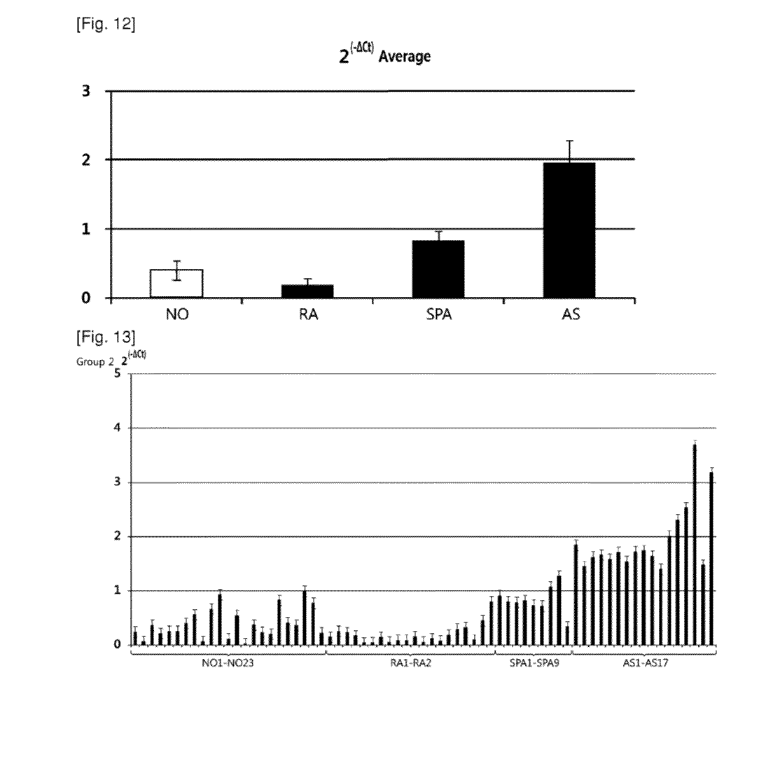

FIG. 11 shows the result of quantitativegroup 2 primer sets, FIG. 12 shows the average of each group of quantitativegroup 2 primer sets, and FIG. 13 shows the result of each sample of quantitativegroup 2 primer sets.

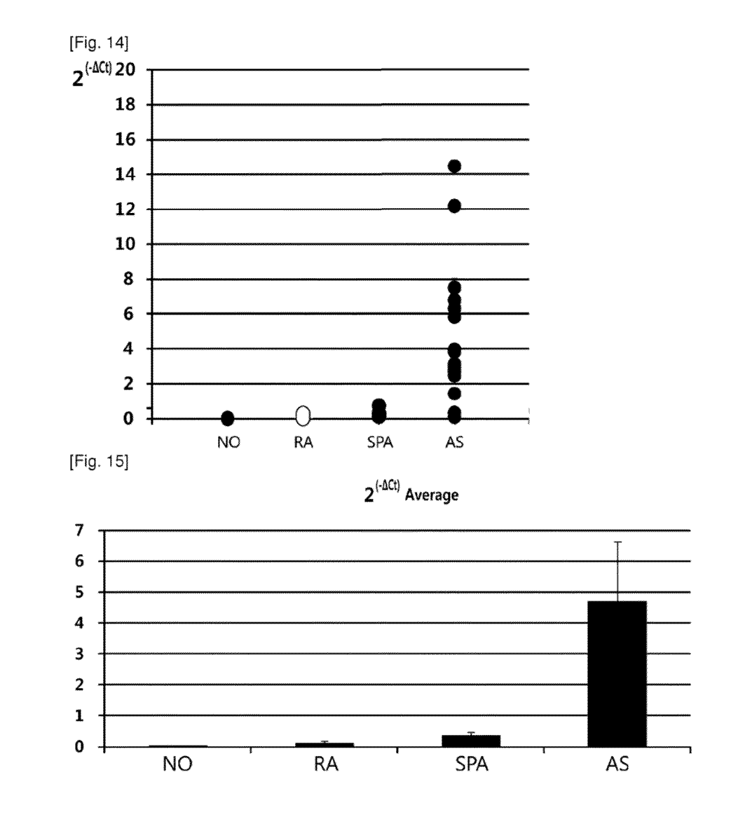

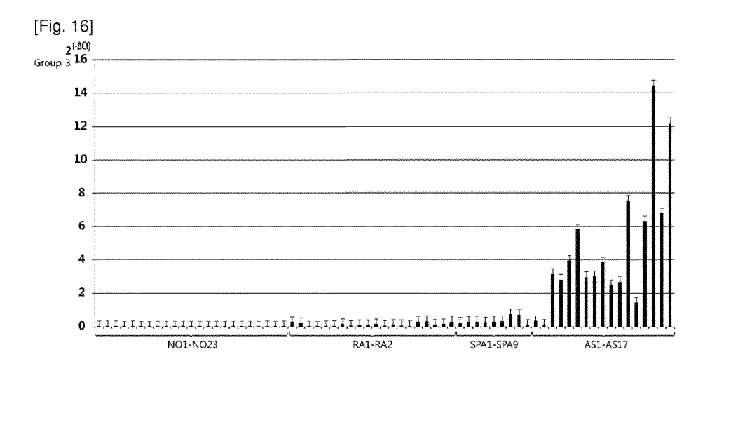

FIG. 14 shows the result of quantitativegroup 3 primer sets, FIG. 15 shows the average of each group of quantitativegroup 3 primer sets, and FIG. 16 shows the result of each sample of quantitative using group 3 primer sets.

As shown in FIGS. 8 to 16, it is clear that the primer sets of the present invention (group 1 to 3) produce specific PCR products in patients with AS.

In conclusion, the primer sets of the present invention can be used for early diagnosis of AS.

### INDUSTRIAL APPLICABILITY

The method for diagnosing AS using the present primer sets and biomarker can be very effective for early diagnosis, tracking progress and prognosis of AS.

1. A primer set for diagnosing Ankylosing Spondylitis: selected from the group consisting of
(a) a forward primer having at least 95% sequence homology with SEQ ID NO: 7;
(b) at least one reverse primer selected from the group consisting of primers of SEQ ID NOs: 9 to 13;
(c) at least one forward primer selected from the group consisting of primers of SEQ ID NOs: 15 to 17;
(d) a reverse primer having at least 95% sequence homology with SEQ ID NO: 19:
(e) a forward primer having at least 95% sequence homology with SEQ ID NO: 18; and
(f) a reverse primer having at least 95% sequence homology with SEQ ID NO: 20.

2-3. (canceled)

4. A method for diagnosing Ankylosing Spondylitis comprising following steps:
(a) synthesizing cDNA by using biological samples of patients;
(b) performing PCR (polymerase chain reacton) by using the above synthetic cDNA and a primer set having at least 80% sequence homology with any one of primer sets of according to claim 1; and
(c) determining nucleic acid sequences of PCR products.

5. The method according to claim 4, the biological sample is selected from the group consisting of saliva, biopsy, blood, skin tissue, liquid cultures, feces, and urine.

6. The method according to claim 5, the biological sample is blood.

7. A kit for diagnosing Ankylosing Spondylitis comprising a primer set having at least 80% sequence homology with any one of primer set according to claim 1.

8. A biomarker for diagnosing Ankylosing Spondylitis comprising total or a part of nucleic acid sequence of SEQ ID NO: 21.

9. A method for diagnosing Ankylosing Spondylitis using total or a part of nucleic acid sequence of SEQ ID NO: 21.

10. The method according to claim 9, the method comprises following steps: (a) synthesizing cDNA by using biological samples of patients; (b) performing PCR (polymerase chain reaction) using the above synthetic cDNA and a primer set having at least 80% sequence homology with any one of primer sets according to claim 1; and (c) identifying total or a part of nucleic acid sequence of SEQ ID NO: 21 in PCR products.

11. The method according to claim 10, the biological sample is selected from the group consisting of saliva, biopsy, blood, skin tissue, liquid cultures, feces, and urine.

12. The method according to claim 11, the biological sample is blood.

13-14. (canceled)
